(19) United States
(12) Patent Application Publication
(10) Pub. No.: US 2022/0010252 A1
(43) Pub. Date: Jan. 13, 2022

(54) MICROPHYSIOLOGICAL CHOROID MODEL

(71) Applicant: Fraunhofer-Gesellschaft zur Förderung der angewandten Forschung e.V., München (DE)

(72) Inventors: Christopher Probst, Stuttgart (DE); Madalena Cipriano, Stuttgart (DE); Peter Loskill, Stuttgart (DE)

(21) Appl. No.: 17/413,381

(22) PCT Filed: Dec. 10, 2019
(86) PCT No.: PCT/EP2019/084406

§ 371 Date: Jun. 11, 2021

(30) Foreign Application Priority Data
Dec. 14, 2018 (DE) 10 2018 221 838.2

(51) Int. Cl. C12M 3/00; C12M 1/00; C12M 1/12; G01N 33/50; C12N 5/071

(52) U.S. Cl. CPC C12M 21/08; C12M 23/34; C12M 25/02; C12N 5/0626; G01N 33/5064; G01N 33/5082; C12M 25/14

(57) ABSTRACT

The invention relates to the field of cultivating biological cells and tissues having an organ-like function on a microphysiological scale and provides a microphysiological reproduction of the choroid and the blood-retinal barrier as an in vitro test system.

The invention relates to the field of cultivating biological cells and tissues having an organ-like function on a microphysiological scale and provides a microphysiological reproduction of the choroid and the blood-retinal barrier as an in vitro test system.

Cell and stem cell-based in vitro models are being developed that can replace ethically problematic and cost-intensive animal models in the research of genetic or idiopathic diseases of the animal or human body and in the development of prophylactic and therapeutic active substances for treating such diseases. It is also important to answer the question of whether and to what extent results found in animal models can be transferred to humans, especially if it has been shown that animal tissues or cells have different structures, cell densities or, at the cellular level, different enzyme or receptor structures so that direct transfer from animal models to humans would actually not be advisable. Here, too, the in vitro model can help if it is possible to reproduce the different cell and tissue properties there and then to be able to compare these properties under controlled conditions.

Microphysiological (MPS) or so-called “Organ-on-a-Chip” (OoaC) systems permit cultivation of isolated animal or human cells. The cells can be derived from defined cell lines, but also from primary cells obtained from human tissue (biopsy) and embryonic origin or from induced pluripotent stem cells (iPS). These cells can then be cultivated under the most physiological conditions possible, for example to reproduce specific tissue types such as lungs, heart, intestines, or kidneys. In the meantime, complex, in particular iPS-based organ systems made up of several cell types, so-called organoids, which can arise largely independently and self-organizing under the influence of a few external signal molecules during in vitro differentiation, have been developed. Examples are retinal organoids that can be cultivated in specially designed microphysiological bioreactors and used as in vitro test systems for the human retina (DE 10 2017 217 738 A). Multi-layer bioreactors with several overlying chambers or channels, optionally separated from one another by semipermeable membranes, for co-cultivating several cell and tissue types are known in principle.

The choriocapillaris is the terminal branching of the choroid of the vertebral eye and forms a vascular layer that faces the retina and that, especially in primates and humans, feeds the outer layers of the retina. The choriocapillaris comprises a fine network of fenestrated capillaries, and, above the basement membrane of the retinal pigment epithelium (RPE), forms a segmented network characterized by end connections. The choriocapillaris is fed with the layer (vascular lamina) from the next larger vessels via lower arterioles and venules. Embedded in connective tissue, it is highly pigmented. The suprachoroid lamina comprises elastic connective tissue and pigmented connective tissue cells. They line the outermost layer of the choroid membrane of the eye. The choroid has neuroectodermal melanocytes which, in addition to synthesizing melanin, also function as part of the immune system. The melanocytes are distributed three-dimensionally over the entire choroid membrane.

Together, the endothelial cells of the blood vessels, which are closely connected to the choroid, and the epithelial cells of the retinal pigment epithelium (RPE), which are closely connected to the choroid, form the so-called blood/retinal barrier, the barrier for the passage of substances from the blood into the retina of the eye, and vice versa. For research into new therapeutic options for certain eye diseases that can be correlated with choroidal function, such as age-dependent macular degeneration (AMD), diabetic retinopathy, especially in patients with type I diabetes, or nearsightedness, findings on the function of the blood/retinal barrier and the interaction of the cell types and tissues involved in the choroid are of great importance. Unfortunately, it has been found that established animal models are particularly unsuitable in this regard or that the knowledge gained with the animal models cannot be easily and directly transferred to the situation in humans. For example, different primates or monkeys have a different tissue structure or different cell densities in the choroid than humans. In addition, genetically determined diseases in humans cannot be well reproduced and investigated in animal models. Recourse to test systems based on genetically diseased human cells would be desirable here.

At the moment there are no known in vitro test systems that can implement the basic functions of the choroid and the blood/retinal barrier in a usable manner. Previous in vitro test systems of the choroid or blood/retinal barrier consist mainly of two-dimensional, 2D monolayers of epithelial cells and endothelial cells which are applied to a semipermeable membrane in a bioreactor in order to imitate a barrier, similar to the blood/retinal barrier, between a simulated blood flow in the bioreactor on the side of the endothelial cells and the epithelial cells.

For example, in the wet form of AMD, new, abnormal blood vessels grow from the choroid under and into the retina. Liquid escapes from these new, leaky vessels, leading to blindness. Groups of the various cell types of the choroid, including melanocytes, are involved in such processes.

It has been found that the choroid has a different density of melanocytes, depending on the species. The density of melanocytes in the human choroid is many times lower than that in other primates or monkeys. The density of choroidal melanocytes also differs many times over between human individuals, similar to pigmentation of the skin.

It is disadvantageous that essential cell types of the choroid, such as melanocytes, are not present in known in vitro models or in vitro test systems.

The present invention was therefore based on the technical problem of providing improved methods and means for establishing physiologically relevant in vitro test systems of the animal or human choroid, in particular the function of the blood/retinal barrier, in particular to establish such in vitro test systems for the choroid, in which melanocytes can also be cultivated in a physiologically similar manner to the in vivo state, and especially in which the melanocytes can be included in different cell densities.

The technical problem is solved by a novel in vitro tissue culture arrangement based on an in particular microphysiological bioreactor and choroid cells, in which melanocytes, even with high cell densities, are cultivated in a three-dimensional arrangement and under physiologically similar extracellular matrix (ECM) to ensure their constant vitality over the duration of the use of the in vitro test system. The subject matter of the invention is characterized in claim 1. This is especially an in vitro tissue culture arrangement which includes or essentially comprises the following elements: a bioreactor with a first chamber, a three-dimensional 3D melanocyte culture arranged in this first chamber, the (isolated) melanocytes which are embedded in a hydrogel. Furthermore, the inventive arrangement has a second chamber of the bioreactor adjoining the first chamber and a first semipermeable membrane which separates the second chamber of the bioreactor from the first chamber of the bioreactor, wherein the membrane side of this first semipermeable membrane facing the first chamber adjoins the 3D melanocyte culture in the first chamber, and in particular is positioned directly adjacent thereto. Furthermore, the inventive arrangement has an in particular confluent first 2D endothelial cell layer localized or arranged in the second chamber of the bioreactor, including (isolated) endothelial cells, wherein this first 2D endothelial cell layer rests against the membrane side of the first semipermeable membrane facing the second chamber, in particular as a single layer or monolayer (monolayer).

In one special embodiment, the inventive arrangement also has in the bioreactor a third chamber of the bioreactor adjoining the second chamber and a second semipermeable membrane which separates this third chamber of the bioreactor from the aforementioned second chamber of the bioreactor, and wherein an in particular confluent second 2D endothelial cell layer, including isolated endothelial cells, is located or arranged in this second chamber of the bioreactor, wherein this second 2D endothelial cell layer rests against the membrane side of the second semipermeable membrane facing the second chamber, also in particular as a monolayer.

In one special variant of this particular embodiment, the inventive arrangement also has a confluent first 2D epithelial cell layer located or arranged in the aforementioned third chamber of the bioreactor and including (isolated) epithelial cells, wherein this 2D epithelial cell layer rests against the membrane side of the second semipermeable membrane facing the third chamber, also in particular as a monolayer.

In special embodiments of these inventive arrangements, the latter also have in the bioreactor a fourth chamber of the bioreactor adjoining the aforementioned first chamber and a third semipermeable membrane which separates this fourth chamber of the bioreactor from the third chamber of the bioreactor, wherein the membrane side of this third semipermeable membrane facing the first chamber is adjacent to the 3D melanocyte culture in the first chamber and in particular rests directly on it, wherein an in particular confluent third 2D endothelial cell layer, including isolated endothelial cells, is located or arranged in this fourth chamber of the bioreactor, wherein this third 2D endothelial cell layer rests against the membrane side of the third semipermeable membrane facing the fourth chamber, also in particular as a monolayer. In the structure of this embodiment of the in vitro tissue culture arrangement, it is preferably provided that the 3D melanocyte culture is embedded between this first semipermeable membrane and the third semipermeable membrane.

The invention therefore particularly provides for cultivating from endothelial cells a 3D melanocyte culture, including or comprising melanocytes embedded in hydrogel, with a 3D structure adjacent to at least one 2D endothelial cell layer, that is, in particular a monolayer. This makes possible a controllable, physiologically adequate interaction between the melanocytes and the endothelial cells, and specific parameters of this cell or tissue interaction can be tested in a targeted manner as an in vitro test system. Physiologically adequate feeding of the cells of the 3D melanocyte culture and the adjacent 2D endothelial cell layer is advantageously made possible in the inventive arrangement. An in vitro test system based on an organ-typical sandwich culture, which reflects the complex structure and function of the choroid in vivo, is thus provided.

According to preferred embodiments of the invention, the in vitro tissue culture arrangement is carried out as a microphysiological reactor, that is, in particular, the chambers in the bioreactor are arranged in layers over one another. In particular, the bioreactor is embodied as a microphysiological bioreactor and the chambers of the bioreactor are embodied as so-called channels or channel structures in the microphysiological bioreactor. Such chambers, channels, or channel structures preferably each have a chamber volume of less than 10 μL, preferably from 1 to 5 μL, on the microphysiological scale.

Advantageously, bioreactor arrangements on a microphysiological scale allow the interaction between cells and tissues in the same dimensions as found in the living organ as an in vitro test system and allow meaningful investigation. The present invention provides for the first time a microphysiological reproduction of the choroid and blood/retinal barrier as an in vitro test system which comes very close to the physiological state in the living organ. In alternative embodiments, however, the invention is not restricted to the microphysiological scale; bioreactors with in part larger chambers, that is, especially chambers with a larger filling volume, can also be provided.

In particular, artificial hydrogels with a defined chemical composition based on dextran crosslinking systems or, alternatively, collagen gels based on collagen or fibronectin gels, are provided as hydrogels. Artificial hydrogels with a defined chemical composition which are preferably provided with additional binding motifs are particularly preferred.

The invention permits, on the one hand, introducing to a microphysiological in vitro test system a defined hydrogel with melanocytes in different cell densities, and, on the other hand, with different stiffnesses, that is, rheological properties, due to the crosslinking strength or protein density of the hydrogel. As a result, the cultivation conditions for the melanocytes in the in vitro test system can be precisely adapted to the in vivo state, be it that the low-melanoma choroid of a person is to be reproduced, or that the influence of different melanocyte densities on the function of the blood/retinal barrier or the immune response in the choroid is to be investigated.

Preferred are the isolated melanocytes which are used for the 3D melanocyte culture used according to the invention and selected from melanocytes isolated directly from human or animal tissue, induced pluripotent stem cells (iPS), and embryonic stem cells. Human embryonic stem cells and, in particular, parts of organs of living humans are excluded.

Preferred are the isolated endothelial cells which are used for the 2D endothelial cell layer used according to the invention and selected from endothelial cells isolated directly from human or animal tissue, induced pluripotent stem cells (iPS), and embryonic stem cells. Microvascular endothelial cells are preferred. Human embryonic stem cells and, in particular, parts of organs of living humans are excluded.

Preferred are the isolated epithelial cells which are used for the 2D epithelial cell layer used according to the invention and selected from epithelial cells isolated directly from human or animal tissue, induced pluripotent stem cells (iPS), and embryonic stem cells. The epithelial cells are particularly preferably retinal pigment epithelial cells (RPE) or epithelial-like cell lines such as ARPE-19. Human embryonic stem cells and, in particular, parts of organs of living humans are excluded.

To produce an inventive microphysiological bioreactor, the layers and channels can be produced by molding polydimethylsiloxane (PDMS) on microstructured silicon wafers. The manufacture of the bioreactor is not limited to this material, however, and other materials such as glass, PC, and PET and combinations thereof are possible. A microstructuring of the respective casting molds (master) is realized in particular by UV lithography, for example, by means of photoresist. The assembly of the bioreactor can take place in several steps: For example, a perfusion channel layer on a carrier film is first applied to a slide glass with a thickness of, for example, 0.17 mmm to 1 mm, in particular after activation in the oxygen plasma, and is pressed on for the mechanical connection. In order to strengthen the connection, this composite material can be heated in a convection oven, for example at 60° C. to 80° C. To create the perfusion channel, the carrier film is then peeled off so that a perfusion channel layer, which ultimately forms one of the chambers of the bioreactor, remains on the carrier glass.

The semipermeable membranes are preferably constructed from materials such as PET. They preferably have a pore size of 4 to 5 μm and a preferred thickness of 10 to 30 μm.

A semipermeable membrane is applied to this chamber or channel layer, for example, as follows: The through-holes for the inflows and outflows in the layers below are created in advance. An in particular functionalized semipermeable membrane is added to the insertion area provided for this purpose. As the next step, another channel layer is placed and pressed on and the entire sandwich is heated to 60° C. to 80° C. in a convection oven, for example for a period of 10 to 24 hours. A plurality of such arrangements produced in layers can be arranged next to one another on a common carrier.

In one further aspect, the invention also provides methods for producing an inventive in vitro tissue culture arrangement. These methods include at least the following steps (c) and (d):

(c) Seeding isolated endothelial cells in a second chamber of a bioreactor, with an orientation of the bioreactor in relation to the gravity vector such that endothelial cells sink onto a membrane side of a first semipermeable membrane facing this second chamber, which membrane separates the second chamber from a first chamber of the bioreactor, and, in particular, adhere there, and,

(d) Cultivating the endothelial cells that have sunk onto this membrane side of the first semipermeable membrane, with the proviso that endothelial cells adhere to this membrane side and grow there, in particular to form a confluent first 2D endothelial cell layer.

These processes according to the invention also include the following steps (g) and (e):

(g) Adding a suspension of isolated melanocytes suspended in a liquid hydrogel precursor to this first chamber of the bioreactor, and,

(h) Allowing the hydrogel precursor to harden to form a hydrogel, so that a 3D melanocyte culture in which isolated melanocytes are embedded in the hydrogel is formed in the first chamber.

These methods according to the invention preferably also include the following steps (e) and (f):

(e) Seeding isolated endothelial cells into this second chamber of the bioreactor, with an orientation of the bioreactor in relation to the gravity vector such that endothelial cells sink onto a membrane side of a second semipermeable membrane facing the second chamber, which membrane separates the second chamber from a third chamber of the bioreactor (100), and, in particular, adhere there, and,

(f) Cultivating the endothelial cells that have sunk onto this membrane side of the second semipermeable membrane (150) so that endothelial cells adhere to this membrane side and grow there, in particular to form a confluent second 2D endothelial cell layer.

The gravity vector is used such that the bioreactor is rotated such that the cells in question can sink along the gravity vector. For this, it is necessary for the cells to be added to the chamber in a suspension in which the cells can sink.

Thus, according to the invention, it is particularly provided that two separate 2D endothelial cell layers are formed in the second chamber or the second channel of the bioreactor, and, on the one hand, oriented in the direction of the 3D melanocyte culture in the adjacent first chamber or first channel, and, on the other hand, oriented in the direction of an adjacent third chamber or third channel, in particular opposite thereto. In this way, the second chamber, which is covered on both sides with a 2D endothelial cell layer, can serve as an in vitro model of a vessel which, on the one hand, is in contact with the melanocytes in the first chamber, and, on the other hand, is in contact with a retinal pigment epithelial layer (RPE) which is preferably present in the third chamber. In this way, active substances to be tested, which would be applied in vivo into the vascular system, that is to say into the bloodstream, can be applied into this second channel of the in vitro tissue culture arrangement in test operations.

In preferred variants, the endothelial cell layers are applied laterally one after the other in the in vitro tissue culture arrangement; in one particularly preferred variant, the endothelial cell layer which is adjacent to the 3D melanocyte culture is applied first. A method is therefore preferred in which steps (c)-(d) are carried out temporally before steps (g)-(h). In one variant, steps (c)-(f) are carried out temporally before steps (g)-(h); in one variant, steps (c)-(d) are carried out temporally before steps (g)-(h), steps (e)-(f) temporally after steps (g)-(h).

Alternatively or additionally, provision is preferably made for seeding an epithelial cell layer in the third chamber of the bioreactor, specifically on the membrane side of the second semipermeable membrane facing the third chamber, especially on the opposite side of the second semipermeable membrane, that is, a 2D endothelial cell layer is arranged or is (yet) seeded on the side facing the second chamber, as explained in the foregoing. The methods according to the invention therefore preferably also include the following steps (a) and (b):

(a) Seeding isolated epithelial cells into the third chamber of the bioreactor, with such an orientation of the bioreactor in relation to the gravity vector that epithelial cells sink onto the membrane side of the second semipermeable membrane facing the third chamber, which membrane side separates the second chamber from a third chamber of the bioreactor, and, in particular, adhere there, and,

(b) Cultivating the epithelial cells that have sunk on this membrane side of the second semipermeable membrane, so that epithelial cells adhere to this membrane side and grow there into an in particular confluent first 2D epithelial cell layer.

In one preferred variant it is provided that steps (a)-(b) are carried out temporally before steps (c)-(h).

In further variants it is additionally provided that a third 2D endothelial cell layer is formed in a fourth chamber of the bioreactor of the inventive in vitro tissue culture arrangement described here, specifically on a third semipermeable membrane that separates this fourth chamber from the first chamber. The colonization of this membrane side of the third semipermeable membrane facing the fourth chamber with endothelial cells is preferably carried out analogously to the procedure described above, particularly preferably also using the operational orientation of the gravity vector, in order to allow the endothelial cells to sink onto this side of the third semipermeable membrane.

One further aspect of the invention relates to in vitro test methods and the use of the inventive in vitro tissue culture arrangement in such test methods. In particular, the interaction of the different cell types and/or the integrity of the barrier, in particular the epithelial barrier and/or the endothelial barrier, is analyzed in the inventive in vitro tissue culture arrangement. This should be done in particular by measuring the substance flows across the semipermeable membranes, by determining electrical parameters (impedance measurement), or by means of solutions of fluorescent labeled macromolecules (e.g. dextran) of different molecular weights to determine the transport rate of the macromolecules across the blood/retinal barrier.

Finally, the tissue from the inventive arrangement can be examined using a histological preparation, in particular for structural changes, but also for changes in the receptor structures. The analysis is carried out in particular using imaging methods such as brightfield, fluorescence, and confocal microscopy and immunohistological staining.

It is also provided that individual intact cells or tissues are gently detached from the inventive arrangement. The detached cells can be supplied to continuous analysis methods such as flow cytometry, or they can be collected for (later) analysis of molecular processes in the detached cells, in particular gene expression. Alternatively or additionally, the analysis of the so-called medium supernatant, which can be obtained and collected from the individual channels of the bioreactor, in particular the endothelial channel, is provided, in particular by means of antibody-based detection methods such as ELISA.

In particular, cellular material, especially immune cells, especially mononuclear cells of the peripheral blood, which are added to least one of the chambers of the inventive arrangement, preferably in the endothelial canal, may also be used as the substance to be tested. With simultaneous or subsequent addition of a substance, the effect of this substance on the onset of the immune response can be examined. One approach is to study the migration of immune cells, especially T cells, from the endothelial channel into the neighboring tissue of melanocytes in hydrogel. Another approach is to investigate whether the immune reaction can be modulated by adding a substance to be tested, which can be demonstrated, for example, by increased migration of immune cells, for example T cells, and/or can be recognized due to increased proliferation of the T cells which are already in the tissue of melanocytes and hydrogel.

In a first approach, the substance to be tested is added to a channel/chamber colonized with the endothelial cells, if necessary after the injection of cell material. This corresponds in particular to the in vivo state of the administration of the substance into the bloodstream. In an alternative approach to such a test method, the substance to be tested is alternatively or additionally added to the channel/chamber colonized with the epithelial cells. In a further alternative approach, the substance to be tested is alternatively or additionally added to the channel/chamber colonized with the melanocytes.

The invention is described in more detail using the following figures and examples without these being limiting.

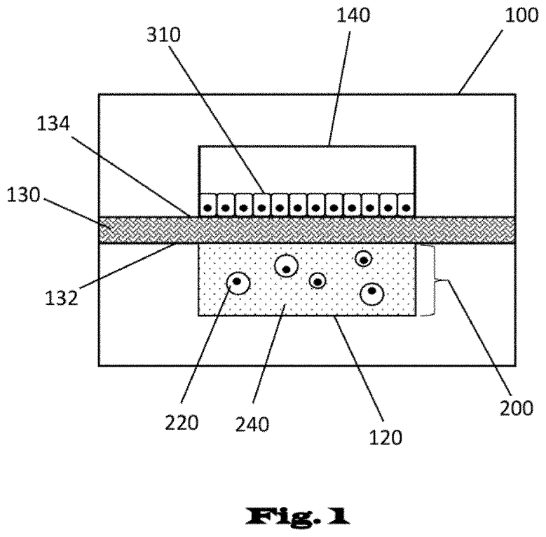

FIG. 1 shows a schematic sectional view of a first embodiment of the inventive in vitro tissue culture arrangement with at least two chambers (120, 140), in which arranged in a first chamber (120) in a bioreactor (100) is a 3D melanocyte culture (200), in which isolated melanocytes (220) are embedded in a hydrogel (240). A second chamber (140) of the bioreactor (100) directly adjoins the first chamber (120). A first semipermeable membrane (130) separates the second chamber (140) from the first chamber (120). It is particularly provided that the membrane side (132) of the first semipermeable membrane (130) facing the first chamber (120) rests against the 3D melanocyte culture (200). A first 2D endothelial cell layer (310) is arranged in the second chamber (140) and rests against the membrane side (134) of the first semipermeable membrane (130) facing the second chamber (140). As a result, the first 2D endothelial cell layer (310) is separated from the 3D melanocyte culture (200) only by the first semipermeable membrane (130), but is connected in a semipermeable manner.

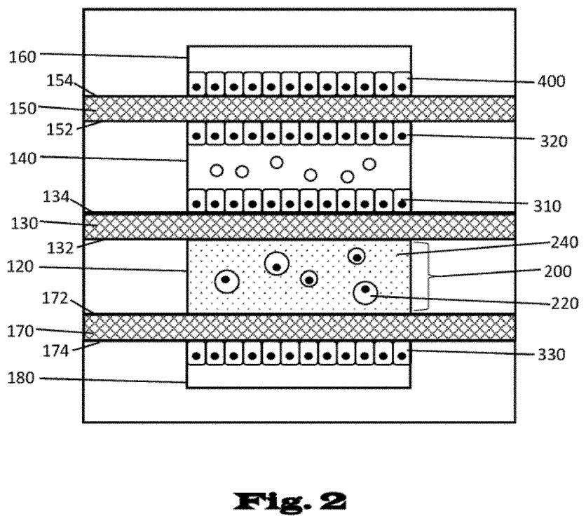

FIG. 2 shows a schematic sectional view of a further embodiment of the inventive in vitro tissue culture arrangement with four chambers (120, 140, 160, 180), in which arranged in the bioreactor (100) in a first chamber (120) is a 3D melanocyte culture (200) in which isolated melanocytes (220) are embedded in a hydrogel (240). A second chamber (140) of the bioreactor (100) directly adjoins the first chamber (120). A first semipermeable membrane (130) separates the second chamber (140) from the first chamber (120). The membrane side (132) of the first semipermeable membrane (130) facing the first chamber (120) rests against the 3D melanocyte culture (200). A first 2D endothelial cell layer (310) is arranged in the second chamber (140) and rests against the membrane side (134) of the first semipermeable membrane (130) facing the second chamber (140). A third chamber (160) of the bioreactor (100) directly adjoins the second chamber (140), specifically on a side of the second chamber (140) opposite the adjoining first chamber (120). A second semipermeable membrane (130) separates the third chamber (160) from the second chamber (140). In this embodiment, a second 2D endothelial cell layer (320) is arranged in the second chamber (140) and rests against the membrane side (152) of the second semipermeable membrane (150) facing the second chamber (140). A 2D epithelial cell layer (400) is also arranged in the third chamber (160) of the bioreactor (100) and rests against the membrane side (154) of the second semipermeable membrane (150) facing the third chamber (160). As a result, the second semipermeable membrane (150) is colonized on both sides and the second 2D endothelial cell layer (320) is separated from the 2D epithelial cell layer (400) by this membrane (150), but connected in a semipermeable manner. In the embodiment shown here with four chambers, in particular a fourth chamber (180) is also formed on the opposite side of the first chamber (120), which is separated from the first chamber (120) by a third semipermeable membrane (170). It is particularly provided that the membrane side (172) of the third semipermeable membrane (170) facing the first chamber (120) rests against the 3D melanocyte culture (200). Arranged in the fourth chamber (180) of the bioreactor (100) is in particular a third 2D endothelial cell layer (330) which rests against the membrane side (174) of the third semipermeable membrane (174) facing the fourth chamber (180). As a result, the third 2D endothelial cell layer (330) is also separated from the 3D melanocyte culture (200) only by the third semipermeable membrane (170), but is connected in a semipermeable manner.

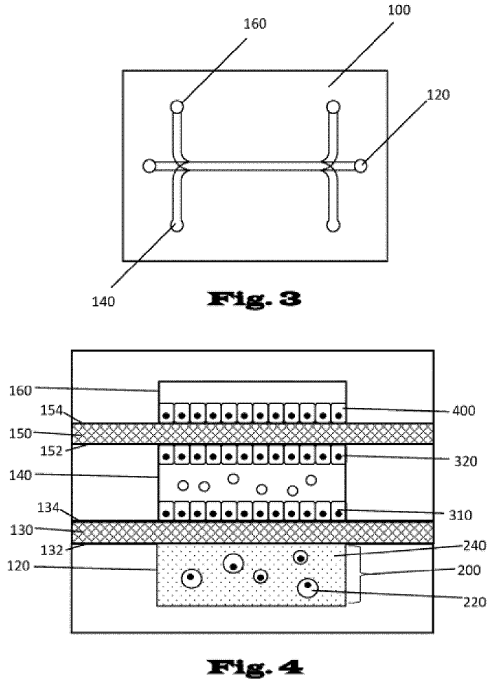

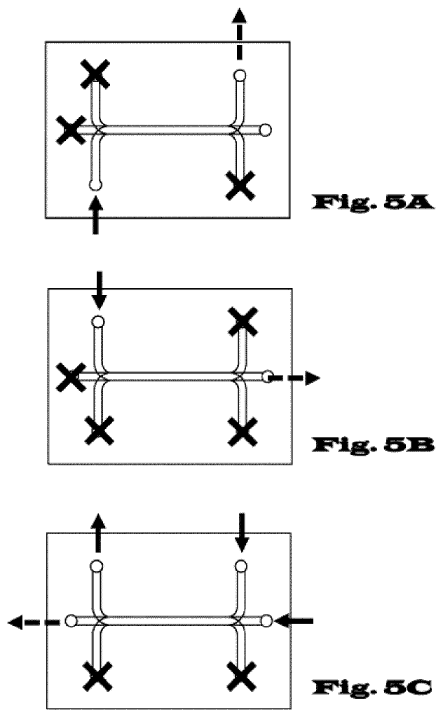

FIG. 3 shows a schematic top view of a typical practical embodiment of the in vitro test system (100) with three channel structures, particularly according to FIG. 4 with one channel (160) for seeding retinal pigment cells, a further channel (140) for seeding endothelial cells, and one channel (130) for loading a hydrogel with melanocytes that is provided there.

FIG. 4 shows a schematic sectional view of one embodiment of the in vitro test system with three channel structures (120, 140, 160) which are separated from one another by two semipermeable membranes (130, 150). In the uppermost channel structure (160), a 2D monolayer of epithelial cells (400), preferably retinal pigment epithelial cells, is applied to the uppermost semipermeable membrane (150). In the case of the channel structure (140) arranged in the center, a 2D monolayer of endothelial cells (310, 320), preferably microvascular endothelial cells, is applied to the underside (152) of the uppermost semipermeable membrane (150) and to the upper side (134) of the lower semi-permeable membrane (130). A hydrogel (240) with melanocytes (220), which forms a 3D melanocyte culture (200), is added to the lowermost channel structure (120) on the underside (132) of the lower semipermeable membrane (130).

FIG. 5 shows schematic top views of the embodiment of the in vitro test system with three channel structures according to FIG. 3 which are partially closed (FIG. 5A) or opened (FIG. 5B) for the different cell types used or can be washed with a constant flow of nutrient medium (FIG. 5C).

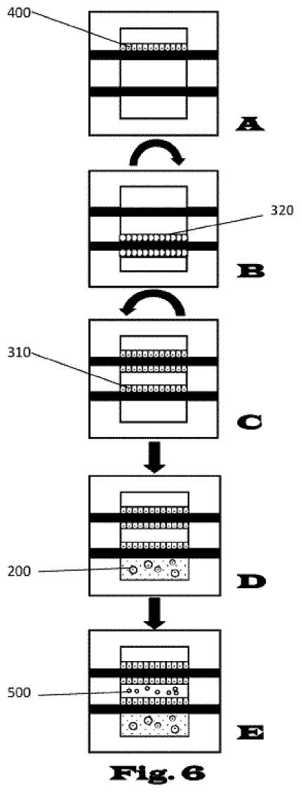

FIG. 6 shows a schematic sectional view of one embodiment of the in vitro test system and the introduction of the various cell types; A: Seeding retinal pigment epithelial cells into the uppermost channel to create a 2D RPE monolayer (400) on top side of the semipermeable membrane; B: Seeding endothelial cells in the center channel, in vitro test system is turned upside down to create a 2D endothelial cell monolayer (320) on the underside of the semipermeable membrane; C: In vitro test system is rotated back to create a second 2D endothelial cell monolayer (310) on the top side of the semipermeable membrane; D: A hydrogel is added to the lower canal and colonized with melanocytes to form the 3D melanocyte culture; E: In the test mode, substances and/or immune cells (500) are applied to the center channel occupied by endothelial cells.

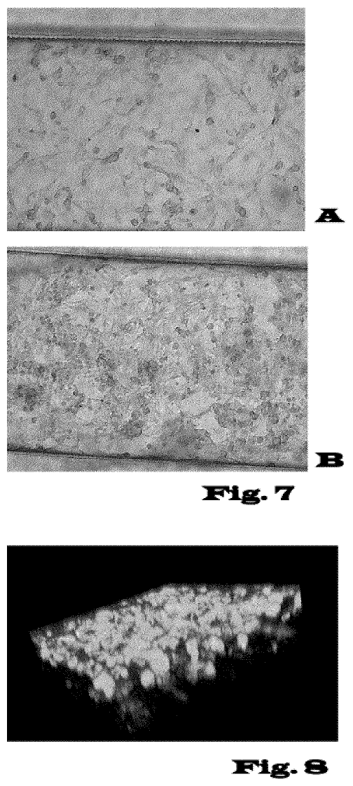

FIG. 7 shows cell densities of melanocytes in a hydrogel in the inventive in vitro arrangement: A: Hydrogel+melanocytes in a cell density that corresponds to that of the human choroid; B: Hydrogel+melanocytes in a cell density that corresponds to the choroid of a primate.

FIG. 8 shows the three-dimensional distribution of hydrogel+melanocytes of the inventive in vitro arrangement, determined and represented by means of the autofluorescence of the melanin formed by the melanocytes.

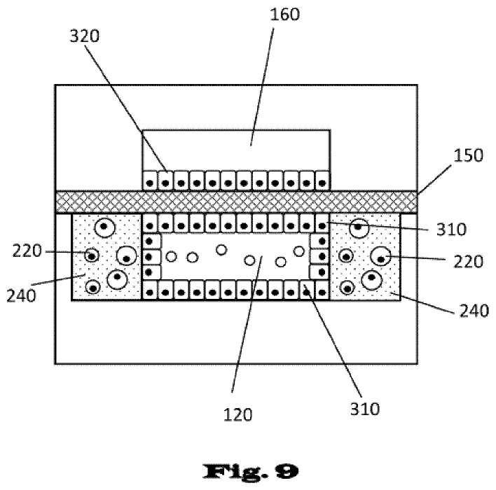

FIG. 9 shows the schematic sectional view of a further embodiment of the in vitro test system with two channel structures (120, 160) which are separated from one another by a semipermeable membrane (150). Endothelial cells or epithelial cells (320) are added to the upper channel (160) as a 2D monolayer. A hydrogel (240) with melanocytes (220) is added to the lower channel (120); endothelial cells (310) are also added to the lower channel (120) and attach to the outside of the hardened hydrogel.

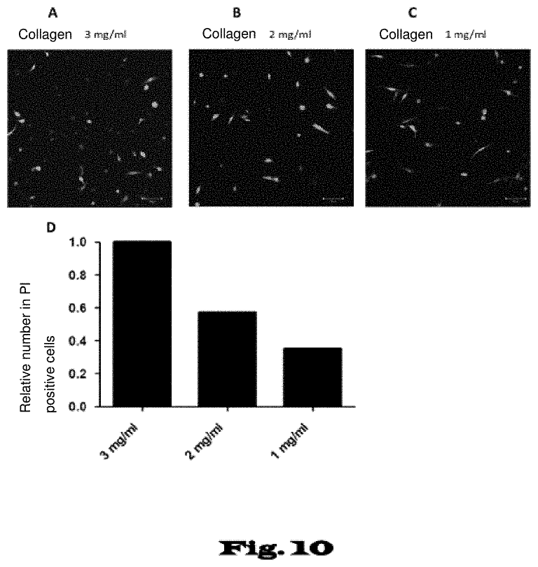

FIG. 10 shows melanocytes embedded in a collagen hydrogel in the inventive in vitro arrangement; the melanocytes have been stained by means of a living/dead stain (vital=fluorescein diacetate, non-vital=propidium iodide (PI)): A: at a concentration of 3 mg/mL; B: at a concentration of 2 mg/mL; C: at a concentration of 1 mg/mL; D shows a bar graph for the cells that are positive (dead) for propidium iodide (PI): The number of PI-positive cells drops significantly as the gel concentration goes down and, conversely, leads to higher vitality.

### PRODUCTION OF A MICROPHYSIOLOGICAL IN VITRO CHOROID TEST SYSTEM

To produce the test system, melanocytes, endothelial cells, and epithelial cells are seeded into a microphysiological bioreactor. The steps are as follows:

1) Seeding of epithelial cells, preferably retinal pigment epithelial cells, in the uppermost channel structure of the bioreactor, said cells forming a 2D monolayer there: For this purpose, the outlet of the endothelial channel of the bioreactor is closed, the outlet of the retinal pigment epithelial channel is closed, the outlet of the melanocyte+hydrogel channel is closed. Cell solution with retinal pigment epithelial cells is flushed into the inlet of the retinal pigment epithelial channel and flushed out via the outlet of the endothelial cell channel.

2) Seeding of endothelial cells, preferably microvascular endothelial cells, in the center channel structure, wherein: a) a first 2D monolayer of said endothelial cells is created on the upper side of the second semipermeable membrane, and b) a second 2D monolayer of said endothelial cells is produced on the lower side of the first semipermeable membrane. For this, the inlet of the endothelial channel is closed, the outlet and inlet of the retinal pigment epithelial channel are closed, and the outlet of the hydrogel+melanocyte channel is closed. Cell solution is flushed into the outlet of the endothelial channel and flushed out via the outlet of the melanocyte channel. A first 2D monolayer is thus created in that a cell solution is flushed into said channel and the in vitro test system is turned upside down to allow the endothelial cells to sink onto the underside of the first semipermeable membrane. The second 2D monolayer is created on the upper side of the second semipermeable membrane by rotating the in vitro test system back after a certain time (15 minutes).

3) Addition of a solution of melanocytes and hydrogel to the lowermost channel structure. The ratio of melanocytes to hydrogel can reproduce the melanocyte cell density of the choroid of humans or primates. The hydrogel can be native ECMs such as collagen, fibronectin, or synthetic hydrogels such as those based on dextran. For this purpose, the inlet and outlet for the retinal pigment epithelial channel are closed. Nutrient medium is flushed into the inlet of the endothelial channel at a constant flow rate (5 μL/hour) and flushed out via the outlet thereof. At the same time, a liquid solution of hydrogel+melanocytes is flushed into the inlet of the melanocyte channel and the outlet thereof is rinsed out. The hydrogel then hardens/solidifies in the channel.

Microphysiological 3D Melanocyte Culture Based on Collagen Hydrogel

Modification of the collagen density/porosity with regard to optimal vitality of the melanocytes as well as the possibility that said cells can adhere to the hydrogel. At a higher collagen concentration (3 mg/ml), vitality decreases sharply, and only a small number of melanocytes can adhere to the gel. This is shown by the fact that these cells are spherical in shape. At lower concentrations (2 mg/ml-1 mg/ml), vitality increases significantly and a greater proportion of the cells can adhere to the hydrogel. An optimal collagen concentration was found at 1 mg/ml, which also has a better viscosity in terms of handling for later flushing into the chip. Higher concentrations of collagen (3 and 2 mg/ml) are difficult to pipette and are therefore flushed into the reactor together with the cells.

1. An in vitro tissue culture arrangement comprising:
a first chamber (120) in a bioreactor (100),
a 3D melanocyte culture (200) arranged in the first chamber (120), in which isolated melanocytes (220) are embedded in a hydrogel (240),
a second chamber (140) in the bioreactor (100) which adjoins the first chamber (120) of the bioreactor (100),
a first semipermeable membrane (130) which separates the second chamber (140) of the bioreactor (100) from the first chamber (120) of the bioreactor (100), wherein the membrane side (132) of the first semipermeable membrane (130) facing the first chamber (120) rests against the 3D melanocyte culture (200), and,
a confluent first 2D endothelial cell layer (310) of isolated endothelial cells which is arranged in the second chamber (140) and rests against the membrane side (134) of the first semipermeable membrane (130) facing the second chamber (140).

2. The in vitro tissue culture arrangement according to claim 1 further comprising:
a third chamber (160) in the bioreactor (100) which adjoins the second chamber (140) of the bioreactor (100),
a second semipermeable membrane (150) which separates the third chamber (160) of the bioreactor (100) from the second chamber (140) of the bioreactor (100), and,
a confluent second 2D endothelial cell layer (320) of isolated endothelial cells which is arranged in the second chamber (140) of the bioreactor (100) and rests against the membrane side (152) of the second semipermeable membrane (150) facing the second chamber (140).

3. The in vitro tissue culture arrangement according to claim 2, further comprising:
a confluent third 2D epithelial cell layer (400) of isolated epithelial cells which is arranged in the third chamber (160) of the bioreactor (100) and rests against the membrane side (154) of the second semipermeable membrane (150) facing the third chamber (160).

4. The in vitro tissue culture arrangement according to claim 1, further comprising:
a fourth chamber (180) in the bioreactor (100) which adjoins the first chamber (120) of the bioreactor (100),
a third semipermeable membrane (170) which separates the fourth chamber (140) of the bioreactor (100) from the first chamber (120) of the bioreactor (100), wherein the membrane side (172) of the third semipermeable membrane (170) facing the first chamber (120) rests against the 3D melanocyte culture (200), and,
a confluent third 2D endothelial cell layer (330) of isolated endothelial cells which is arranged in the fourth chamber (180) of the bioreactor (100) and rests against the membrane side (174) of the third semipermeable membrane (170) facing the fourth chamber (180).

5. The in vitro tissue culture arrangement of claim 4, wherein the 3D melanocyte culture (200) is embedded between the first semipermeable membrane (130) and the third semipermeable membrane (170).

6. The in vitro tissue culture arrangement according to claim 1, wherein the chambers (120, 140, 160, 180) in the bioreactor (100) are arranged layered directly one above the other.

7. The in vitro tissue culture arrangement according to claim 6, wherein the bioreactor (100) is designed as a microphysiological bioreactor and the chambers (120, 140, 160, 180) are designed as channel structures with a chamber volume of less than 10 μL each.

8. A method for producing an in vitro tissue culture arrangement according to claim 1, comprising the steps:
c) Seeding isolated endothelial cells into a second chamber (140) of a bioreactor (100), with such an orientation of the bioreactor (100) in relation to the gravity vector that endothelial cells sink onto a membrane side (134) of a first semipermeable membrane (130) facing the second chamber (140), which membrane separates the second chamber (140) from a first chamber (120) of the bioreactor (100),
d) Cultivating the endothelial cells that have sunk onto this membrane side (134) of the first semipermeable membrane (130) so that endothelial cells adhere to this membrane side (134) and grow there to form a confluent first 2D endothelial cell layer (310), and,
g) Adding a suspension of isolated melanocytes (220) in liquid hydrogel precursor to the first chamber (120) of the bioreactor (100), and,
h) Allowing the hydrogel precursor to harden to form a hydrogel (240), so that a 3D melanocyte culture (200) in which isolated melanocytes (220) are embedded in the hydrogel (240) is formed in the first chamber (120).

9. The method according to claim 8, further comprising the steps:
e) Seeding isolated endothelial cells into a second chamber (140) of the bioreactor (100), with such an orientation of the bioreactor (100) in relation to the gravity vector that endothelial cells sink onto a membrane side (152) of a second semipermeable membrane (150) facing the second chamber (140), which membrane separates the second chamber (140) from a third chamber (160) of the bioreactor (100), and,
f) Cultivating the endothelial cells that have sunk onto this membrane side (152) of the second semipermeable membrane (150) so that endothelial cells adhere to this membrane side (152) and grow there to form a confluent second 2D endothelial cell layer (320).

10. The method according to claim 9, wherein steps (c)-(f) are carried out temporally before steps (g)-(h).

11. The method according to claim 9, further including the steps:
a) Seeding isolated epithelial cells into the third chamber (160) of the bioreactor (100), with such an orientation of the bioreactor (100) in relation to the gravity vector that epithelial cells sink onto the membrane side (154) of the second semipermeable membrane (150) facing the third chamber (160), which membrane separates the second chamber (140) from a third chamber (160) of the bioreactor (100), and,
b) Cultivating the epithelial cells that have sunk onto this membrane side (154) of the second semipermeable membrane (150) so that epithelial cells adhere to this membrane side (154) and grow there to form a confluent first 2D epithelial cell layer (400).

12. The method according to claim 11, wherein steps (a)-(b) are carried out temporally before steps (c)-(h).

13. The method for in vitro testing of the modulatory effect of a substance on the function of the blood/retinal barrier, comprising the steps:
Providing the in vitro tissue culture arrangement according to claim 1,
Adding the substance to at least one chamber (120, 140, 160, 180) of this in vitro tissue culture arrangement,
Registering and detecting changes in the function of the blood/retinal barrier after the substance has been added compared to the state before the substance was added, wherein the characteristic value determined for the function of the blood/retinal barrier is selected from: macromolecule Transport Rate and Electrical Impedance (TEER).

14. The method for in vitro testing of the modulatory effect of a substance on the immune reaction in the choroid, comprising the steps:
Providing the in vitro tissue culture arrangement according to claim 1,
Adding immune cells to a chamber (140, 180) of this in vitro tissue culture arrangement that carries endothelial cells,
Adding the substance to at least one chamber (120, 140, 160, 180) of this in vitro tissue culture arrangement,
Registering and detecting the immune reaction after the substance has been added, the immune reaction being selected from: Migration of the immune cells from the endothelial cell layer into the neighboring 3D melanocyte culture and proliferation of the immune cells in the 3D melanocyte culture.
